(19) United States
(12) Patent Application Publication
(10) Pub. No.: US 2009/0118736 A1
(43) Pub. Date: May 7, 2009

(54) Apparatus and Method for Aligning a Guide Pin for Joint Re-Surfacing

(76) Inventor: Stefan Kreuzer, Houston, TX (US)

Correspondence Address: CONLEY ROSE, P.C.;David A. Rose, P. O. BOX 3267, HOUSTON, TX, 77253-3267, US

(21) Appl. No.: 12/265,224

(22) Filed: Nov. 5, 2008

Related U.S. Application Data
(60) Provisional application No. 60/985,547, filed on Nov. 5, 2007.

(51) Int. Cl. A61B 17/58

(52) U.S. Cl. 606/96

(57) ABSTRACT

A drill guide for aligning a guide pin. In an embodiment, the drill guide comprises a base having a concave inner surface, an outer surface, and a central axis perpendicular to the inner surface. In addition, the drill guide comprises a drilling template extending axially from the outer surface of the base along the central axis. The drilling template includes a plurality of through-bores. Further, the drill guide comprises a first curved member extending along a central longitudinal axis from a fixed end integral with the base to a free end distal the base, and a second curved member extending along a central longitudinal axis from a fixed end integral with the base to a free end distal the base. The first curved member and the second curved member are angularly spaced about 180° apart relative to the central axis and include an elongate locking slot.

### CROSS-REFERENCE TO RELATED APPLICATIONS

This application claims the benefit of U.S. provisional application Ser. No. 60/985,547, filed Nov. 5, 2007, and entitled “Joint Re-Surfacing Drill Guide”,” which is hereby incorporated herein by reference in its entirety.

### STATEMENT REGARDING FEDERALLY SPONSORED RESEARCH OR DEVELOPMENT

Not Applicable.

### BACKGROUND

1. Field of the Invention

The invention relates generally to devices and methods for orthopedic surgery. More particularly, the invention relates to devices and method for aligning a guide pin for re-surfacing the ball portion of a ball and socket joint.

2. Background of the Invention

Total hip replacement, also referred to as hip arthroplasty, is a surgical procedure in which the hip joint is replaced by a prosthetic implant. Hip joint replacement surgery is typically conducted to relieve arthritis or to repair severe joint damage resulting from fracture. Hip resurfacing arthroplasty is a type of hip replacement that replaces the surface of the joint but removes far less bone than the traditional total hip replacement. Since hip resurfacing removes less bone, it may be particularly suited for younger patients that are expected to need a second, or revision, hip replacement surgery as they grow older and wear-out or out-grow the original artificial hip replacement.

Although hip resurfacing arthroplasty removes less bone, it is a technically more difficult operation with a higher complication rate then routine total hip replacement. One potential complication is femoral neck fracture, which is generally influenced by four factors including varus placement of the femoral component, notching during surgery, Body Mass Index (BMI), and gender. Although BMI and gender of a given patient cannot be controlled by the surgeon, varus placement of the femoral component and notching during surgery may be impacted by the surgeon's performance and procedure. In particular, proper placement of the guide pin to resurface the femoral head can reduce notching and improve varus placement of the femoral component.

Optimal placement of the guide pin depends on the anteversion/retroversion (generally “version”) angular orientation of the guide pin relative to the ball portion of the femoral neck, the varus/valgus orientation of the guide pin relative to the ball portion of the femoral neck, and the intersection of the femoral neck central axis with the surface of the ball portion of the femoral neck. As is known in the art, varus/valgus alignment generally refers to the angular orientation of the femur longitudinal axis measured in the coronal or frontal plane, and femoral version generally refers to the angular orientation of the femoral neck measured in the transverse plane. Referring briefly to FIG. 1, an exemplary femur 10 includes a shaft 20 having a longitudinal axis 25, and a femoral neck 30 extending from the upper end of shaft 20. Femoral neck 30 has a ball portion 32 adapted to fit within a mating socket or acetebaular cup in the pelvis to form the ball and socket hip joint. Ball portion 32 has an approximate geometric center 38 about which ball portion and femur 10 generally rotate. In addition, femoral neck 30 has a central axis 35 that passes through geometric center 38 of ball portion 32. A first axis 45 passes through center 38 generally perpendicular to central axis 35 of femoral neck 2, and a second axis 55 passes through center 38 generally orthogonal to both axes 35, 45. In general, rotation about first axis 45 contributes to a variation in the version angle of the femoral neck and a variation in the varus/valgus orientation of the femur, and a rotation about second axis 55 contributes to a variation in the version angle of the femoral neck and a variation in the varus/valgus orientation of the femur. In general, first axis 45 may be any axis perpendicular to femoral neck axis 35, and second axis 55 may be any axis orthogonal to axes 35, 45. Rotation about any such set of orthogonal axes will vary both the version angular orientation and the varus/valgus angular orientation. Due to the subtleties involved with aligning version angle, the varus/valgus angle, and guide pin entry point during resurfacing operations due to numerous anatomical variations, most conventional drill guides are bulky, cumbersome and require alignment of all three dimensions simultaneously, which can be quite challenging and often requires repositioning the guide pin.

Accordingly, there is a need for devices and methods that enable a surgeon to properly position a guide pin for joint resurfacing procedures. Such devices and methods would be particularly well received if they provided a relatively simple, cost effective approach to position the guide pin without requiring simultaneous varus/valgus alignment, the entry point of the guide pin, and anteversion/retroversion alignment.

### BRIEF SUMMARY OF SOME OF THE PREFERRED EMBODIMENTS

These and other needs in the art are addressed in one embodiment by a drill guide for aligning a guide pin. In an embodiment, the drill guide comprises a base having a concave inner surface, an outer surface opposite the inner surface, and a central axis perpendicular to the inner surface. In addition, the drill guide comprises a drilling template extending axially from the outer surface of the base along the central axis and includes a plurality of through-bores extending completely through the drilling template to the inner surface of the base. Further, the drill guide comprises a first curved member extending along a central longitudinal axis from a fixed end integral with the base to a free end distal the base and a second curved member extending along a central longitudinal axis from a fixed end integral with the base to a free end distal the base. The first curved member and the second curved member are angularly spaced about 180 degrees apart relative to the central axis and include an elongate locking slot aligned with the central longitudinal axis.

These and other needs in the art are addressed in another embodiment by a drill guide for aligning a guide pin. In an embodiment, the drill guide comprises a base having a concave inner surface, an outer surface opposite the inner surface, and a central axis perpendicular to the inner surface. In addition, the drill guide comprises a drilling template centered on the upper surface of the base and includes a plurality of through-bores extending completely through the drilling template to the inner surface of the base. Further, the drill guide comprises a plurality of curved members extending along a central longitudinal axis from a fixed end integral with the base to a free end distal the base and angularly spaced relative to the central axis. Each curved member includes an elongate locking slot aligned with the central longitudinal axis.

These and other needs in the art are addressed in another embodiment by a method for aligning a guide pin for re-surfacing a joint. In an embodiment, the method comprises providing a drill guide. The drill guide includes a base having a curved inner surface, an outer surface opposite the inner surface, and a central axis perpendicular to the inner surface. In addition, the drill guide includes a plurality of curved members extending from the base and angularly spaced relative to the central axis. Each curved member extends along a central longitudinal axis from a first end coupled to the base to a second end distal the base. Further, the drill guide includes a drilling template centered on the upper surface of the base. The drilling template includes a plurality of through-bores. Each curved member includes an elongate locking slot positioned along the central longitudinal axis. In addition, the method comprises placing the drill guide over a ball portion of a ball and socket joint such that the inner surface of the base engages the ball portion. Further, the method comprises restricting the rotation of the drill guide relative to a first axis. Moreover, the method comprises restricting the rotation of the drill guide relative to a second axis orthogonal to the first axis.

Thus, embodiments described herein comprise a combination of features and advantages intended to address various shortcomings associated with certain prior devices. The various characteristics described above, as well as other features, will be readily apparent to those skilled in the art upon reading the following detailed description of the preferred embodiments, and by referring to the accompanying drawings.

The foregoing has outlined rather broadly certain features and technical advantages of the disclosed devices and methods in order that the detailed description that follows may be better understood. Additional features and advantages will be described hereinafter. It should be appreciated by those skilled in the art that the conception and the specific embodiments disclosed may be readily utilized as a basis for modifying or designing other structures for carrying out the same purposes of the invention. It should also be realized by those skilled in the art that such equivalent constructions do not depart from the spirit and scope of the invention as set forth in the appended claims.

### BRIEF DESCRIPTION OF THE DRAWINGS

For a detailed description of the preferred embodiments of the invention, reference will now be made to the accompanying drawings in which:

FIG. 1 is a front view of a femur;

FIG. 2 is a perspective view of an embodiment of a drill guide constructed in accordance with the principles described herein;

FIG. 3 is a perspective view illustrating the placement of the drill guide of FIG. 1 on the ball portion of a hip joint in preparation for a resurfacing procedure;

FIG. 4 is a perspective view of an embodiment of a drill guide constructed in accordance with the principles described herein;

FIG. 5 is a perspective view of another embodiment of a drill guide constructed in accordance with the principles described herein;

FIG. 6 is a bottom perspective view of the drill guide of FIG. 5;

FIG. 7 is a perspective view of another embodiment of a drill guide constructed in accordance with the principles described herein and including a removably coupled drilling template;

FIG. 8 is a top view illustrating an embodiment of a drill guide constructed in accordance with the principles described herein and including a movably coupled drilling template; and

FIG. 9 is a top view of a drill guide in accordance with the principles described herein and including a double-axis adjustable drilling template.

### DETAILED DESCRIPTION OF THE PREFERRED EMBODIMENTS

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Certain terms are used throughout the following description and claims to refer to particular features or components. As one skilled in the art will appreciate, different persons may refer to the same feature or component by different names. This document does not intend to distinguish between components or features that differ in name but not function. The drawing figures are not necessarily to scale. Certain features and components herein may be shown exaggerated in scale or in somewhat schematic form and some details of conventional elements may not be shown in interest of clarity and conciseness.

In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection, or through an indirect connection via other devices and connections. Further, the terms “radial” and “radially” may be used to described positions, movement, or distances perpendicular to a central axis or longitudinal, while the terms “axial” and “axially” may be used to describe positions, movement, or distances parallel to the central or longitudinal axis.

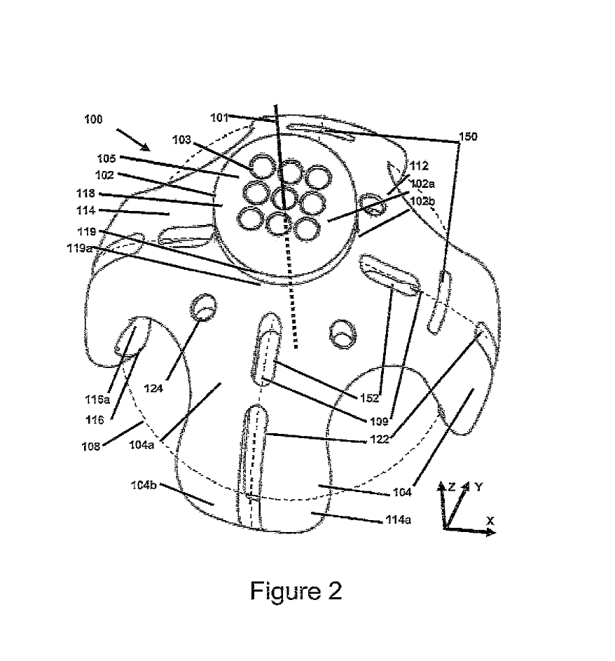

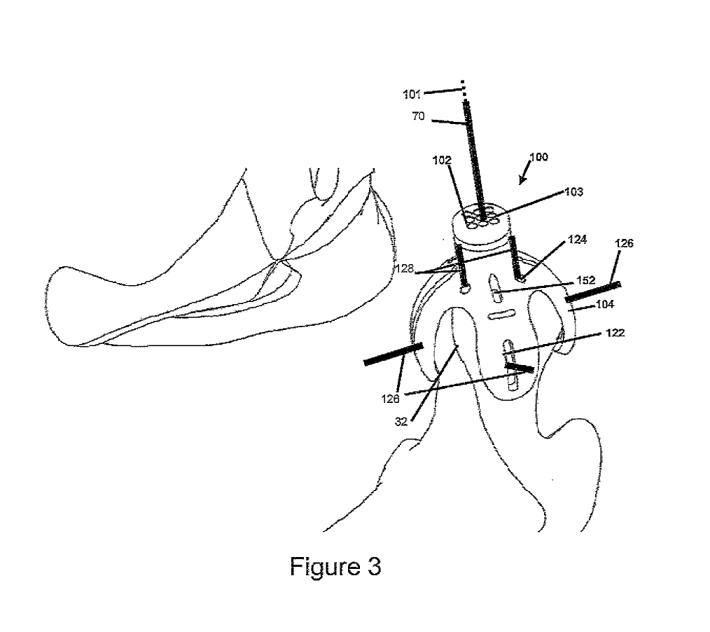

Referring now to FIG. 2, an embodiment of a drill guide 100 for alignment of a guide pin for ball joint re-surfacing is shown. Drill guide 100 includes a generally thin-walled curvilinear base 112 having a central axis 101 and a plurality of thin-walled curvilinear members or arms 104 extending generally radially outward and axially downward from base 112. Together, curved members 104 and base 112 form a cup-, or acetabular-, shaped structure adapted to fit snugly over the head of the ball portion of a ball and socket joint (e.g., FIG. 3). As will be described in more detail below, drill guide 100 facilitates the alignment of a guide pin for purposes of resurfacing the ball portion of a ball and socket joint.

Base 112 has a partially spherical geometry including a convex upper or outer surface 114 and a concave lower or inner surface 116 substantially parallel with outer surface 114. Inner surface 116 may generally be described as being disposed on a reference sphere 108. A central axis 101 passes through the center of base 112 substantially perpendicular to inner and outer surfaces 114, 116. Still further, base 112 includes a plurality of locking through holes or bores 124, each locking hole 124 radially positioned between the outer radial periphery of base 112 and a drilling template 102 disposed at the center of base 112 (i.e., centered relative to central axis 101). In this embodiment, locking holes 124 are uniformly angularly spaced about 90 degrees apart about central axis 101. Further, each locking hole 124 is positioned in base 112 between each pair of curved members 104. In general, each locking hole 124 may have any suitable diameter, but is preferably has a diameter between about 2 mm and 6 mm. Locking holes 124 provide a means to restrict the movement and rotation of drill guide 100 relative to the ball portion on which it is disposed for resurfacing operations. In particular, a pin is disposed through one or more holes 124 and into positive engagement with the ball portion to restrict translational and rotational movement of drill guide 100 relative to the ball portion.

Referring still to FIG. 2, drilling template 102 is disposed at the center of base 112 (i.e., centered relative to central axis 101) and extends axially, relative to central axis 101, from outer surface 114 of base 112. Drilling template 102 has a free end 102a distal base 112 comprising a planar surface 118 generally perpendicular to axis 101, and a fixed end 102b integral with base 112. A generally cylindrical surface 119 extends between ends 102a, b of drilling template 102. In this embodiment, an annular recess or groove 119a is provided about cylindrical surface 119 proximal free end 102a. Groove 119a offers the potential to enhance the ability to grip and manipulate drill guide 100. Although drilling template 102 shown in FIG. 2 is generally cylindrical, in other embodiments, the drilling template (e.g., drilling template 102) may have any other suitable shape including, without limitation, rectangular, hexagonal, or octagonal.

Drilling template 102 also includes a plurality of substantially parallel through-bores 103 extending from planar surface 118 of free end 102a to inner surface 116 of base 112. In this embodiment, each through-bore 103 is substantially parallel to central axis 101. However, in other embodiments one or more of the through-bores (e.g. through-bores 103) may be oriented at an acute angle relative to a central axis (e.g. central axis 101). Further, in this embodiment, each bore 103 is formed by a rigid, durable insert sleeve 105 intended to reduce and/or prevent damage to drilling template 102 during guide pin insertion operations. Sleeves 105 may comprise any suitable rigid, durable material including, without limitation, metals or metal alloys (e.g., stainless steel, aluminum, etc.), polymer (e.g., polyethylene), composite, or combinations thereof. Preferably, sleeve 105 comprises a rigid durable, low-friction polymer such as polyethylene.

In general, through-bores 103 may have any suitable diameter, spacing, and arrangement. However, each through-bore (e.g., through-bore 103) preferably has a diameter between about 1 mm and about 10 mm, more preferably between about 2 mm and 5 mm, and even more preferably between about 2 mm to about 3 mm. Further, the through-bores are preferably spaced less than or equal to about 2 mm apart measured by the least distance between the outer perimeters of two adjacent through-bores (e.g., through-bores 103). It should be appreciated that the spacing of the through-bores may be less than 0, in which case the outer perimeter of two adjacent through-bores may overlap or cross. In the embodiment shown in FIG. 2, drilling template 102 includes nine through-bores 103 arranged in a three-by-three square pattern. Each through-bore 103 has a diameter of about 2 to 3 mm, and every pair of adjacent through-bores 103 are spaced at least 1 to 2 mm apart.

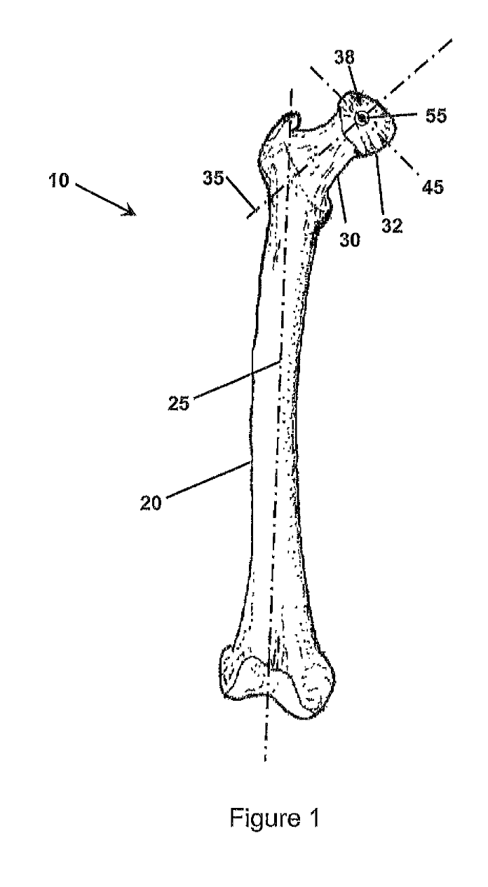

During ball joint resurfacing, drill guide 100 is placed over the ball portion to be resurfaced and through-bores 103 provide a path for a guide pin to be inserted into the ball portion for the resurfacing procedures. Depending on a variety of factors including, without limitation, user preferences, bone structure and geometry, patient BMI, patient gender, and expected patient activities and associated loading, a specific through-bore 103 may be selected to optimize joint resurfacing geometry. In this manner, drilling template 102 allows alignment of a guide pin with an axis passing through the ball portion that may be offset or misaligned with the center of the ball portion itself. For example, the drilling template permits alignment of a guide pin with the central axis of the femoral neck (e.g., central axis 35 of femoral neck 30 previously described with reference to FIG. 1), which may not be centered on the surface of the ball portion of the femoral neck (e.g., point of intersection 38 as previously described with reference to FIG. 1 may not be centered on ball portion 32).

Although drilling template 102 is shown and described as extending axially from base 102, in other embodiments, the drilling template (e.g., drilling template 102) may be flush with the outer surface of the base. In such embodiments, the drilling template does not extend from the outer surface of the base (e.g., outer surface 114 of base 112), and the through-bores of the drilling template (e.g., through-bores 103) extend from the outer surface of the base to the inner surface of the base (e.g., inner surface 116). Further, although drilling template 102 is integral with base 112 in the embodiment shown in FIG. 2, in other embodiments, the drilling template (e.g., drilling template 102) may be a separate component that is coupled to the base. For example, the drilling template may be rotatably coupled to the base such that the drilling template may be rotated clockwise and/or counterclockwise about the central axis of the base (e.g., axis 101) to provide yet another degree of freedom to orient a guide pin relative to the ball portion. As another example, the drilling template may be removably coupled to the base such that different drilling templates having different drilling patterns may be interchangeable on a particular base and drill guide.

Referring still to FIG. 2, as previously described, each curved member or arm 104 extends radially outward and axially downward from base 112 to form an acetabular-shaped structure. More specifically, each curved member 104 extends along a central longitudinal axis 109 from a fixed end 104a integral with base 112 to a free end 104b distal base 112. A projection of longitudinal axis 109 of each curved member intersects central axis 101. In this embodiment, four symmetrically spaced curved members 104 are provided. In particular, curved members 104 are uniformly angularly spaced about 90° apart about axis 101.

Each curved member 104 has a convex, partially spherical outer surface 114a, a concave, partially spherical inner surface 116a generally parallel with outer surface 114a and disposed on reference sphere 108 previously described. Outer surface 114a of each curved member 104 is contiguous with and continuously contoured with outer surface 114 of base 112. Likewise, inner surface 116a of each curved member 104 is contiguous with and continuously contoured with inner surface 116 of base 112. Since inner surface 116a of each curved member 104 and inner surface 116 of base 112 are each disposed on a common reference sphere 108, base 112 and curved members 104 form a partially spherical drill guide 100.

Referring still to FIG. 2, each curved member 104 further comprises an elongate locking slot 122 extending completely through its respective curved member 104 from outer surface 114a to inner surface 116a. Each slot 122 is centered on, and extends along, central longitudinal axis 109 of its respective curved member 104. It should be appreciated that slots 122 do not extend completely to ends 104a, b. Locking slots 122 provide a means for restricting rotation of drill guide 100 about central axis 101. In particular, during a resurfacing procedure, drill guide 100 is disposed on the ball portion to be resurfaced and oriented as desired, and then one or more slot pin 126 is passed through one or more slots 122 into engagement with the ball portion, thereby restricting rotation of drill guide about central axis 101. Although one or more slot pin 126 passing through one or more slots 122 and into engagement with the ball portion restricts rotation of drill guide 100 about central axis 101, the slots 122 are free to move relative to the one or more slot pin 126 along longitudinal axis 109. Thus, drill guide 100 may be rotated about an axis perpendicular to central axis 101 and passing through either pair of opposing slots 122 (i.e., slots 122 angularly spaced 180 degrees apart relative to central axis 101).

Referring still to FIG. 2, two adjacent curved members 104 (i.e., two curved members angularly spaced 90 degrees apart relative to central axis 101) each include a depth of resection indication slot 150 disposed between slot 122 and fixed end 104a and oriented substantially perpendicular to elongate slot 122. Depth of resection inspection slot 150 permits measurement and inspection of the ball portion during resurfacing procedures.

Drill guide 100 also includes a plurality of inspection slots 152, each aligned but axially spaced above one of locking slots 122 (relative to central longitudinal axis 109). In particular, each inspection slot 152 extends along central longitudinal axis 109 from the upper portion of curved member 104, across fixed end 104a, and into base 112. Although inspection slots 152 extend into base 112, each is radially spaced apart from drilling template 112 (relative to central axis 101). Each inspection slot 152 extends completely from outer surfaces 114, 114a to inner surfaces 116, 116a, thereby providing a means to view and inspect the ball portion and soft tissue disposed within drill guide 100.

Referring now to FIG. 3, drill guide 100 offers the potential for improved orientation of a guide pin for resurfacing of the ball portion of a ball and socket joint. As shown in FIG. 3, drill guide 100 is utilized to position a guide pin for resurfacing of the ball portion of a hip joint. In particular, drill guide 100 is placed snugly over the ball portion of the joint, with inner surfaces 116, 116a facing the ball portion and central axis 101 roughly aligned with femoral neck axis 35. Due to the configuration and design of drill guide 100, once it is placed on ball portion 32, a first axis 45 perpendicular to the femoral neck axis 35 inherently passes through locking slots 122 of one pair of opposed curved members 104 (i.e., one pair curved members spaced 180 degrees apart), and a second axis 55 orthogonal to axes 35, 45 inherently passes through locking slots 122 of the other pair of opposed curved members 104. As previously described, axes 45, 55 may be any pair of axes orthogonal to each other and femoral neck axis 35.

Once drill guide 100 is sufficiently disposed on ball portion 32, one or more slot pins 126 are disposed through locking slots 122 of one pair of opposed curved members 104 and into positive engagement with ball portion 32, and one or more pins are disposed through locking slots 122 of the other pair of opposed curved members 104. As shown in FIG. 3, two slot pins 126 disposed about 180 degrees apart are preferably disposed through an axial mid-portion (relative to longitudinal axis 109) of locking slots 122 of one pair of opposed curved members 104 and into engagement with ball portion 32, and two slot pins 126 disposed about 180 degrees apart are preferably disposed through an axial mid-portion (relative to longitudinal axis 109) of locking slots 122 of the other pair of opposed curved members 104 and into engagement with ball portion 32. As a result, the rotation of drill guide 100 about center 38 is restricted to rotation about first axis 45 and second axis 55.

With the rotation of drill guide 100 restricted to two degrees (i.e., about axes 45, 55), the orientation of drill guide 100 is adjusted to achieve the desired position of drilling template 112 relative to ball portion 32. To reduce complexities of simultaneous adjustment of drill guide 100 about multiple axes (e.g., axes 45, 55), drill guide 100 is preferably initially rotated about first axis 45, followed by second axis 55, or vice versa. It should be appreciated that slot pins 126 slidingly engage locking slots 122, and thus, are permitted to slide through locking slots 122 as the orientation of drill guide 100 is adjusted about axes 45, 55. Once the desired rotation about axes 45, 55 and orientation of drill guide 100 and drilling template 112 is achieved, one or more hole pins 128 or other suitable securing means are inserted through one or more locking hole 124 and into positive engagement with ball portion 32, thereby completely restricting rotation of drill guide 100 and drilling template 112 about center 38.

With the position of drilling template 112 locked relative to ball portion 32, a guide pin 70 is advanced through one of the through-bores 103 for subsequent resurfacing operations. As previously described, an array of through-bores 103 are provided in drilling template 112. Consequently, to accommodate for any variation in the final orientation and locking of the drilling template 112 relative to ball portion 32, multiple through-bores 103, each providing a slightly different entry point is available to achieve the desired guide pin 70 orientation.

During placement of drill guide 100 on ball portion 102, pinning of drill guide 100 to ball portion 102, adjustment of drill guide 100 relative to ball portion 102, and locking of drill guide 100 relative to ball portion 32, inspection slots 152 may be utilized by the surgeon or user of device 100 to view ball portion 32 and associated soft tissue.

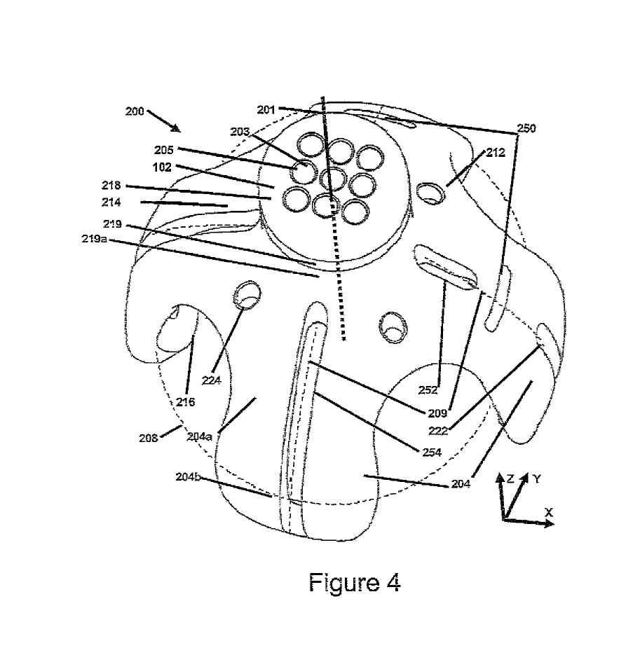

Referring now to FIG. 4, another embodiment of a drill guide 200 is shown. Drill guide 200 is substantially the same as drill guide 100 previously described. Namely, drill guide 200 includes a base 212 with a central axis 201, and a plurality of curved members 204 as previously described. Base 212 includes a plurality of uniformly angularly spaced locking bores or through-holes 224. In addition, a drilling template 202 as previously described extends axially from an outer surface 214 of base 212 (relative to central axis 201).

Two adjacent curved members 204 (i.e., two curved members angularly spaced 90 degrees apart relative to central axis 201) each include a depth of resection indication slot 250 disposed between an elongate locking slot 222 and a fixed end 204a, and oriented substantially perpendicular to locking slot 222. Further, the two adjacent curved members 204 including resection indication slots 250 each include an inspection slot 252 that is aligned with but axially spaced above one of locking slots 222 (relative to central longitudinal axis 109). However, unlike drill guide 100 previously described with reference to FIG. 2, in this embodiment, the two adjacent curved members 204 that do not include a resection slot 250 include only one combined slot 254 extending along central longitudinal axis 209 from distal free end 204b, across fixed end 204a, and into base 212. In other words, in this embodiment, a separate and distinct inspection slot 252 is not provided on these curved members 204.

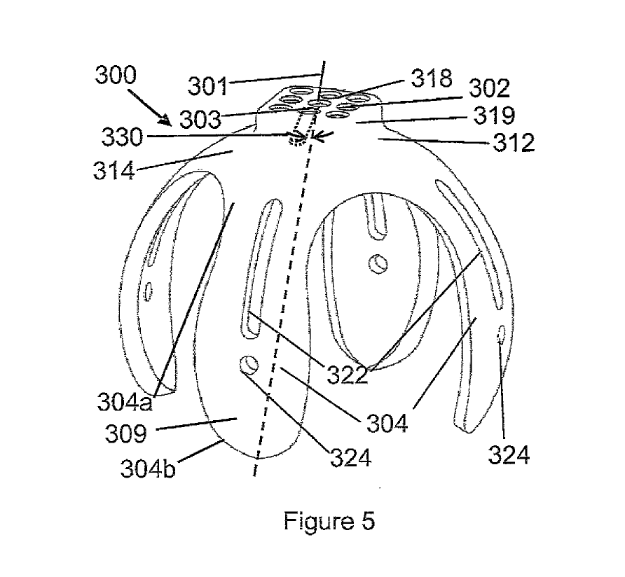

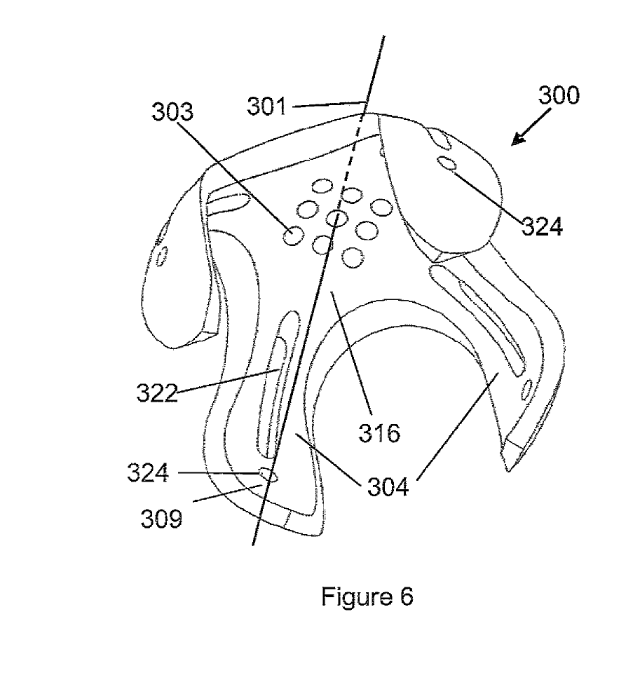

Referring now to FIGS. 5 and 6, another embodiment of a drill guide 300 is shown. Drill guide 300 is similar to drill guide 100 previously described. Namely, drill guide 300 includes a base 312 with a central axis 301, and four uniformly angularly spaced curved members 304 extending radially outward and axially downward from base 312. A drilling template 302 is centered on the outer surface 314 of base 312 and extends axially along central axis 301 from the outer surface 314 of base 312. Each curved member 304 extends along a central longitudinal axis 309 from a fixed end 304a integral with base 312 to a free end 304b distal base 312. Further, each curved member 304 includes an elongate locking slot 322 extending along longitudinal axis 309. However, unlike drill guide 100 previously described, in this embodiment, drilling template 302 has a generally rectangular outer surface 309 extending between its distal planar surface 318 and base 312. Further, at least one of through-bores 303 extending through drilling template 302 is oriented at an acute angle 330 relative to the central axis 301. In this particular embodiment, angle 330 is about 10°. Further, in this embodiment, no separate and distinct inspection slots (e.g., inspection slots 152 previously described with reference to FIG. 2) or resection indication slots (e.g., resection indication slots 150 previously described with reference to FIG. 2) are provided. Further, in this embodiment, base 312 does not include locking through-bores or holes. Rather, a locking through-bore or hole 324 is provided in each curved member 304 between locking slot 322 and free end 304b, generally disposed along longitudinal axis 309.

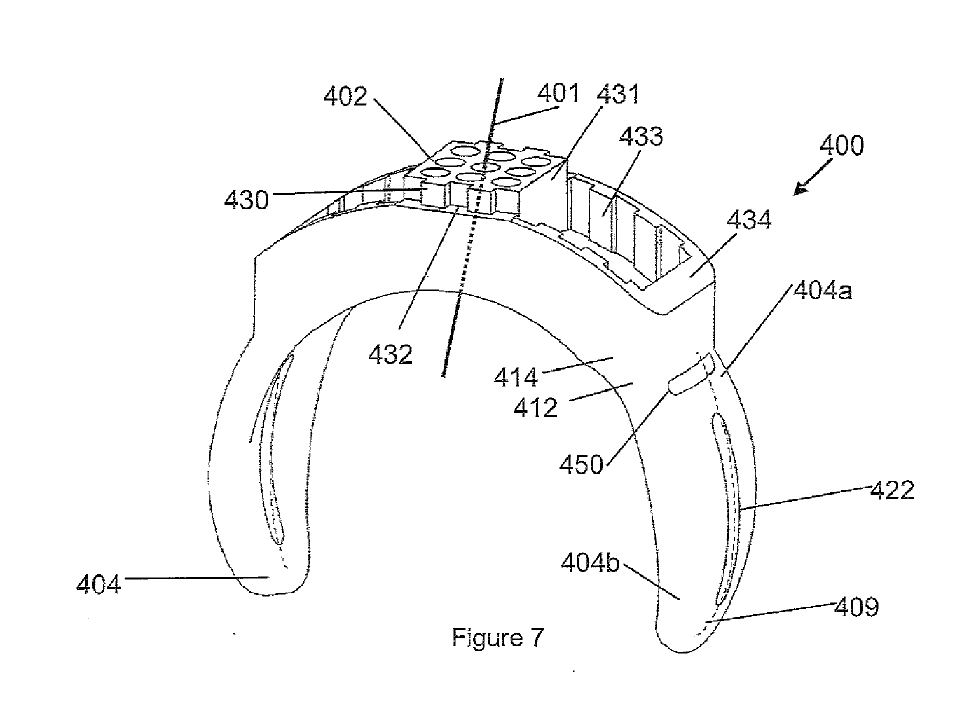

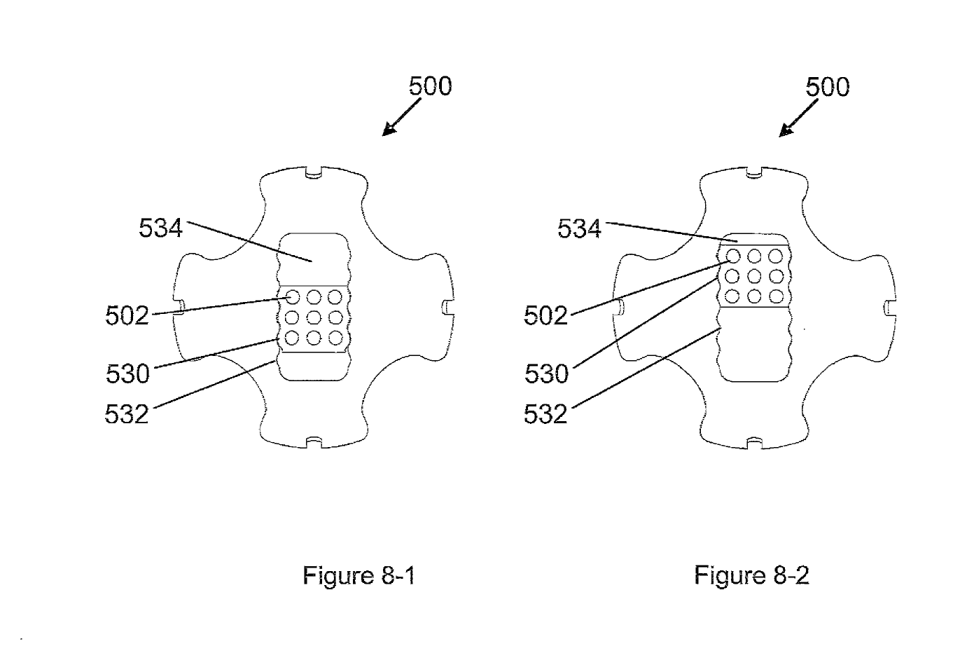

Referring now to FIG. 7, another embodiment of a drill guide 400 is shown. Drill guide 400 includes a base 412 with a central axis 401 and a plurality of curved members 404 extending radially outward and axially down from base 412. However, in this embodiment, only two curved members 404, spaced 180 degrees apart are provided. Each curved member 404 extends along a central longitudinal axis 409 from a fixed end 404a integral with base 412 to a free end 404b distal base 412. Further, each curved member includes an elongate slot 422 disposed along longitudinal axis 409 between ends 404a, b. Unlike drill guides 100, 200, 300 previously described, in this embodiment, a drilling template 402 provided on base 412 is adjustable. In particular, drilling template 402 is removably coupled to a drilling template receptacle or housing 434 via a series of mating teeth 430, 432 provided on the engaging outer surface 431 of drilling template 402 and inner surface 433 of housing 434, respectively. Drilling template housing 434 is integral with and extends axially from outer surface 414 of base 412. Thus, drilling template 402 may be inserted into housing 434 by properly aligning mating teeth 430, 432. Employment of a removable drilling template enables the option of using interchangeable drilling templates with different sized through-bores, different through-bore spacing, different through-bore pattern, or combinations thereof. Further, as elongate housing 434 has a length L₄₃₄ that is greater than the length L₄₀₂ of drilling template 402, the position of drilling template 402 along the length L₄₃₄ of housing 434 may be varied as desired. In this manner, the position of drilling template 402 may be adjusted, without relocating drill guide 400, by removing the drilling template 402 from the drill guide receptacle 434 and reinserting the drilling template 402 into a different location within the drill guide receptacle 434. Although mating teeth 430, 432 are shown as substantially rectangular in this embodiment, in general, the mating teeth may have any suitable mating configuration. For example, as shown in FIG. 8, mating teeth 530, 532 provided on removably coupled drilling guide 502 and drilling guide receptacle 534, respectively are curved. The curved teeth geometry shown in FIG. 8 may permit sliding adjustment of the drilling template 502 without requiring drilling template 502 to be removed from the drill guide receptacle 534.

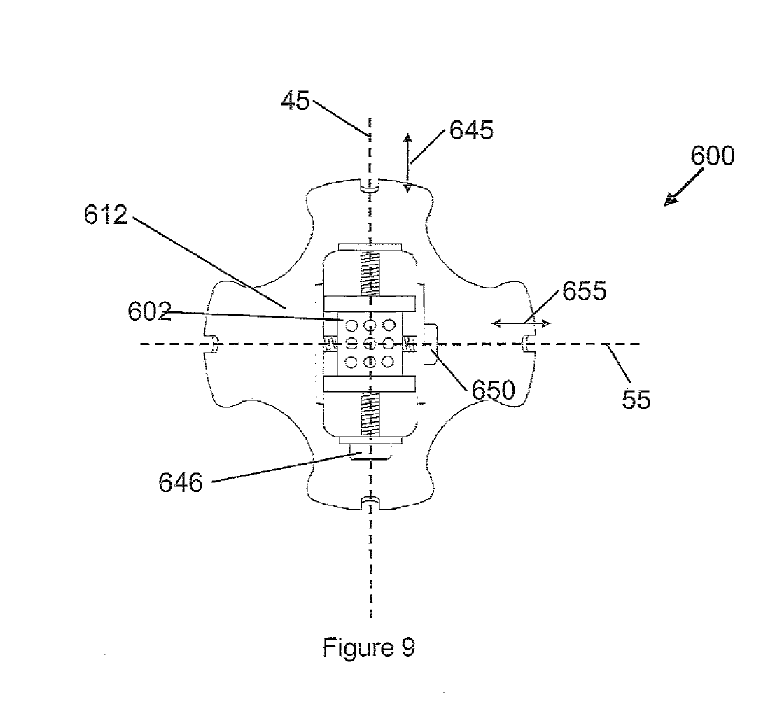

Referring now to FIG. 9, another embodiment of a drilling guide 600 is shown. Drilling guide 600 is substantially the same as drilling guide 100 previously described with the exception that drilling guide includes a drilling template 602 that is adjustably coupled to base 612 of drilling guide 600 such that drilling template 602 may be moved in a first direction 645 generally parallel to first axis 45 and/or in a second direction 655 generally parallel to second axis 55. In particular, adjustable drilling template 602 is disposed within a drilling template housing or receptacle 634 integral with and extending axially from the upper surface of base 612. The position of drilling template 602 within receptacle 634 is adjusted by a first worm drive 646 that adjusts drilling template 612 in first direction 645 and a second worm drive 650 that adjusts drilling template 602 in the second direction 655.

Embodiments of drill guide 100, 200, 300, 500, and 600 may comprise any suitable material(s) including without limitation metals (e.g., stainless steel, titanium, etc.), non-metals (e.g., polymer, composites, etc.) or combinations thereof. The components of drill guide 100, 200, 300, 500, and 600 are preferably manufactured from a durable biocompatible material such as titanium, stainless steel, or polymers such as high density polyethylene. A polymeric drill guide 100, 200, 300, 500, and 600 offers the potential for relatively inexpensive material and manufacturing costs, thereby allowing for an economically feasible disposable drill guide 100, 200, 300, 500, and 600.

Moreover, the components of drill guide 100, 200, 300, 500, and 600 may be manufactured by any suitable methods. Examples of suitable methods include, without limitation, casting or molding, machining, laser cutting, electromechanical deposition (EMD), or combinations thereof. The components of drill guide 100, 200, 300, 500, and 600 may be assembled by any suitable method including without limitation welding, press fitting, or combinations thereof. Moreover, it is envisioned that various sizes of drill guide 100, 200, 300, 500, and 600 may be manufactured to accommodate different sized joints. For example, women and children may have different joint sizes than an adult male joint.

Although embodiments described herein were described for use in positioning a guide pin for resurfacing the ball portion of a hip ball and socket joint, in general, embodiments of drill guide 100, 200, 300, 500, and 600 may be sized and utilized to position a guide pin for resurfacing the ball portion of any ball and socket joint.

While embodiments of the invention have been shown and described, modifications thereof can be made by one skilled in the art without departing from the spirit and teachings. The embodiments described and the examples provided herein are exemplary only, and are not intended to be limiting. Many variations and modifications of the embodiments disclosed herein are possible. Accordingly, the scope of protection is not limited by the description set out above, but is only limited by the claims which follow, that scope including all equivalents of the subject matter of the claims.

What is claimed is:

1. A drill guide for aligning a guide pin comprising:
a base having a concave inner surface, an outer surface opposite the inner surface, and a central axis perpendicular to the inner surface;
a drilling template extending axially from the outer surface of the base along the central axis, wherein the drilling template includes a plurality of through-bores extending completely through the drilling template to the inner surface of the base;
a first curved member extending along a central longitudinal axis from a fixed end integral with the base to a free end distal the base;
a second curved member extending along a central longitudinal axis from a fixed end integral with the base to a free end distal the base;
wherein the first curved member and the second curved member are angularly spaced about 180 degrees apart relative to the central axis;
wherein each curved member includes an elongate locking slot aligned with the central longitudinal axis.

2. The drill guide of claim 1 further comprising:
a third curved member extending along a central longitudinal axis from a fixed end integral with the base to a free end distal the base;
a fourth curved member extending along a central longitudinal axis from a fixed end integral with the base to a free end distal the base;
wherein the third curved member and the fourth curved member are angularly spaced about 180 degrees apart relative to the central axis; and
wherein the third curved member is angularly spaced about 90 degrees from the first curved member and the fourth curved member is angularly spaced about 90 degrees from the second curved member.

3. The drill guide of claim 2 wherein a projection of each central longitudinal axis intersects the central axis.

4. The drill guide of claim 3 wherein the base comprises a first through-hole circumferentially disposed between the longitudinal axis of the first curved member and the longitudinal axis of the third curved member.

5. The drill guide of claim 4 comprising a second through-hole circumferentially disposed between the longitudinal axis of the second curved member and the longitudinal axis of the third curved member.

6. The drill guide of claim 1 wherein the inner surface of the base is a partially spherical surface, and wherein each curved member has a concave partially spherical inner surface contiguous with the inner surface of the base.

7. The drill guide of claim 2 wherein the first curved member and the third curved member each include an elongate slot positioned between the locking slot and the fixed end, and substantially perpendicular to the central longitudinal axis.

8. The drill guide of claim 7 further comprising:
a first elongate inspection slot extending from the second curved member to the base, wherein the first elongate inspection slot is aligned with the locking slot of the second curved member and axially spaced from the locking slot of the second curved member relative to the central longitudinal axis of the second curved member;
a second elongate inspection slot extending from the fourth curved member to the base, wherein the second elongate inspection slot is aligned with the locking slot of the fourth curved member and axially spaced from the locking slot of the fourth curved member relative to the central longitudinal axis of the fourth curved member.

9. The drill guide of claim 2 wherein the first curved member includes a locking through hole disposed between the locking slot of the first curved member and the free end of the first curved member.

10. The drill guide of claim 1 wherein each through-bore in the drilling template is formed by a rigid cylindrical insert.

11. The drill guide of claim 1 wherein the drilling template is integral with the base.

12. The drill guide of claim 1 wherein the drilling template is removably coupled to the base.

13. The drill guide of claim 2 wherein the position of the drilling template is adjustable relative to the base along a first axis extending between the locking slot of the first curved member and the locking slot of the second curved member, and wherein the position of the drilling template is adjustable relative to the base along a second axis extending between the locking slot of the third curved member and the locking slot of the fourth curved member.

14. The drill guide of claim 1 wherein at least one of the plurality of through-bores is oriented substantially parallel to the central axis.

15. A drill guide for aligning a guide pin comprising:
a base having a concave inner surface, an outer surface opposite the inner surface, and a central axis perpendicular to the inner surface;
a drilling template centered on the upper surface of the base, wherein the drilling template includes a plurality of through-bores extending to the inner surface of the base;
a plurality of curved members extending from the base and angularly spaced relative to the central axis, wherein each curved member extends along a central longitudinal axis from a fixed end integral with the base to a free end distal the base;
wherein each curved member includes an elongate locking slot aligned with the central longitudinal axis.

16. The drill guide of claim 15 wherein the plurality of curved members includes four curved members angularly spaced about 90 degrees apart relative to the central axis, and wherein a projection of the central axis of each curved member intersects the central axis.

17. The drill guide of claim 16 wherein the base further comprises a plurality of through holes, each through hole extending from the outer surface of the base to the inner surface and being positioned between a pair of adjacent curved members.

18. The drill guide of claim 16 wherein each curved member includes a concave inner surface contiguous with the inner surface of the base, wherein the inner surface of each curved member and the inner surface of the base are substantially disposed along a common reference sphere.

19. The drill guide of claim 16 wherein a pair of adjacent curved members each include an elongate slot positioned between the locking slot and the fixed end, and substantially perpendicular to the central longitudinal axis.

20. The drill guide of claim 16 wherein the drilling template extends axially from the outer surface of the base.

21. A method of aligning a guide pin for re-surfacing a joint comprising:
(a) providing a drill guide comprising:
a base having a curved inner surface, an outer surface opposite the inner surface, and a central axis perpendicular to the inner surface;
a plurality of curved members extending from the base and angularly spaced relative to the central axis, wherein each curved member extends along a central longitudinal axis from a first end coupled to the base to a second end distal the base;
a drilling template centered on the upper surface of the base, wherein the drilling template includes a plurality of through-bores;
wherein each curved member includes an elongate locking slot positioned along the central longitudinal axis;
(b) placing the drill guide over a ball portion of a ball and socket joint such that the inner surface of the base engages the ball portion;
(c) restricting the rotation of the drill guide relative to a first axis; and
(d) restricting the rotation of the drill guide relative to a second axis orthogonal to the first axis.

22. The method of claim 21 wherein a first pair of the curved members are angularly spaced about 180 degrees apart relative to the central axis, and a second pair of the curved members are angularly spaced about 180 degrees apart relative to the central axis and angularly spaced about 90 degrees from the first pair of curved members.

23. The method of claim 22 wherein (c) comprising passing a pin through the elongate slot of each curved member in the first pair of curved members and into positive engagement with the ball portion.

24. The method of claim 23 wherein (d) comprises passing a pin through the elongate slot of each curved member in the second pair of curved members and into positive engagement with the ball portion.

25. The method of claim 21 further comprising:
(e) rotating the drill guide about the first axis;
(f) rotating the drill guide about the second axis;
(g) fixing the orientation of the drill guide relative to the ball portion.

26. The method of claim 25 wherein the drill guide further comprises at least one locking hole, and wherein (g) comprises passing a pin through the locking hole and into positive engagement with the ball portion.

27. The drill guide of claim 15 wherein at least one of plurality of through-bores is oriented substantially parallel to the central axis.
